(19) United States
(12) Patent Application Publication
(10) Pub. No.: US 2024/0366659 A1
(43) Pub. Date: Nov. 7, 2024

(54) Activated Carbon for Detoxification of Intoxications, Including Methods of Use Thereof

(71) Applicant: AllPet Inc. DBA Dr. Cuddles, Bethesda, MD (US)

(72) Inventors: Mathieu Glassman, Bethesda, MD (US); Jonathan Daniel Foster, Bethesda, MD (US)

(73) Assignee: AllPet Inc. DBA Dr. Cuddles, Bethesda, MD (US)

(21) Appl. No.: 18/656,476

(22) Filed: May 6, 2024

Related U.S. Application Data
(60) Provisional application No. 63/500,232, filed on May 4, 2023.

(51) Int. Cl. A61K 33/44

(52) U.S. Cl. CPC A61K 33/44

(57) ABSTRACT

Activated carbon compounds for the detoxification of intoxications, including methods of use thereof are provided. Many embodiments provide an approach to detoxifying individuals, including household pets (e.g., dogs and/or cats) from toxic substances. Many embodiments are directed to compositions including activated carbon compounds that can be used to detoxify toxins and other intoxications in an individual. Many embodiments may use activated carbon spheres for detoxification. Many embodiments of compositions can be administered orally. In numerous embodiments, the individual is a human and/or a non-human animal, such as a household pet, including dogs and/or cats.

### CROSS-REFERENCE TO RELATED APPLICATIONS

The current disclosure claims the benefit, under 35 U.S.C. § 119(e), of U.S. Provisional Patent Application No. 63/500,232 entitled “Activated Carbon for Detoxification of Intoxications, Including Methods of Use Thereof” filed May 4, 2023. The disclosure of U.S. Provisional Patent Application No. 63/500,232 is hereby incorporated by reference in its entirety for all purposes.

### FIELD OF THE INVENTION

The present invention relates to veterinary medicaments for detoxification, more specifically, compositions including activated carbon spheres to detoxify animals that have ingested harmful chemicals.

### BACKGROUND

Drug, food, and toxin poisoning at home are a common and serious clinical problem in dogs and cats. Such substances include drugs, chemicals, some human foods, pesticides, and household cleaners, among others. These toxic substances can cause severe harm to the animal's health, and in some cases, even death. Because many drugs and toxins ingested by pets at home do not have specific antidote for the treatment of poisoning, gastric decontamination techniques are employed to treat overdoses or poisoning by preventing the further absorption of drug. The timely detoxification of these animals is critical to their survival, and the existing methods have limitations.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

Some embodiments of the invention include a method to detoxification and gastrointestinal decontamination comprising: dosing an amount of activated carbon compounds; and providing the dosed amount of activated carbon compounds to an individual, wherein the individual is a mammal.

In some embodiments, the activated carbon compounds comprise activated carbon spheres.

In some embodiments, the activated carbon compounds consist of 100% activated carbon spheres.

In some embodiments, the individual is a human or a household pet.

In some embodiments, the individual is a dog or cat.

In some embodiments, the individual has consumed or is suspected of consuming or having consumed a toxic substance.

Some embodiments further comprise identifying the individual based on one or more of behavioral, symptomatic, and evidentiary information.

In some embodiments, the behavioral information includes one or more of: whining, pacing, panting, and crying; the symptomatic information includes one or more of: pain, vomiting, diarrhea, and constipation; and the evidentiary information includes one or more of: spilled substances, partially eaten items, and empty wrappers.

In some embodiments, the toxic substance is selected from one or more intoxicants in Table 1.

In some embodiments, dosing an amount of activated carbon compounds comprises measuring an amount of activated carbon compounds to provide to the individual.

In some embodiments, dosing an amount of activated carbon compounds further comprises mixing the measured amount of activated carbon compounds with a substance to ease consumption.

In some embodiments, the substance is selected from: food, drink, peanut butter, canned food, water, broth, and softened food.

In some embodiments, providing the dosed amount of activated carbon compounds comprises forcibly providing the dosed amount to the individual by gastric intubation or gavage.

Some embodiments further comprise monitoring the individual for changes in behavior or symptoms.

Some embodiments further comprise: if symptoms or behavior do not improve or if symptoms or behavior worsen, dosing a second amount of activated carbon compounds; and providing the second dosed amount of activated carbon compounds to the individual.

Some embodiments include a composition for detoxification, comprising a container including an amount of activated carbon compounds.

In some embodiments, the activated carbon compounds comprise activated carbon spheres.

Some embodiments further comprise an edible ingredient, in which the activated carbon compounds are mixed.

In some embodiments, the edible ingredient is selected from the group consisting of: a dog biscuit, a wet treat, a gelatinous treat, a cat treat, a human food, a snack, and a beverage.

In some embodiments, the container is a multi-part container forming a first pouch and a second pouch, wherein the first pouch includes the amount of activated carbon compounds, while the second pouch includes an edible ingredient.

In some embodiments, the container includes a plurality of doses of activated carbon compounds.

In some embodiments, the container comprises markings denoting individual doses of activated carbon compounds.

Some embodiments include a detoxification kit, comprising: a box packaging a container including an amount of activated carbon compounds.

Some embodiments further comprise at least one of: a measuring device, a mixing device, and a dish.

In some embodiments, the activated carbon compounds comprise activated carbon in a resin matrix.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides an example of a multi-part envelope or packaging in accordance with various embodiments.

FIG. 2 provides an example of a method for providing ACSs to an individual in accordance with various embodiments.

### DETAILED DESCRIPTION

Toxicity can come from any number of avenues including accidental and intentional ingestion. Common intoxicants and/or toxic agents, in veterinary medicine, include (but are not limited to) prescription and over-the-counter medications, veterinary medications, health supplements, contaminated foods, household goods (e.g., cleaning agents, insecticides, pesticides, rodenticides, herbicides, etc.), poisonous plants, and outdoor or industrial products (e.g., antifreeze, ethylene glycol, gasoline, paint thinner, etc.). Common toxins for children include (but are not limited to) cosmetics, personal care products, cleaning substances, laundry products, pain medications (both prescription and over-the-counter medications), topical preparations, vitamins and supplements, antihistamines, pesticides, plants, and/or antimicrobials. Common toxins for adults include (but are not limited to) pain medications (both prescription and over-the-counter medications), sedatives, hypnotics, antipsychotics, antidepressants, cardiovascular drugs, cleaning substances, alcohols, pesticides, anticonvulsants, cosmetics, and/or personal care products. For many animals, foods that are safe for human consumption may be toxic, such as grapes, raisins, chocolate, onions, among others. Table 1 provides a non-limiting list of exemplary and common sources of intoxicants to many non-human animals.

One of the primary ways to decontaminate poisoned animals is via emesis induction such as using an emesis agent. Emesis induction is a gold standard procedure for gastric decontamination in the veterinary patient if it is deemed safe based on the toxin ingested. However, safe and effective emesis agents may not be available at home for pets such as dogs and/or cats. For example, cats should have emesis induction performed by the veterinarian with the use of alpha₂-adrenergic agonists (e.g., xylazine, dexmedetomidine). In dogs, the use of hydrogen peroxide can be recommended for at-home emesis, and a centrally acting opioid such as apomorphine is used in hospital. But the appropriate toxicology history and the dosages of emesis agent should be known before performing emesis induction. (See, e.g., Justin A. Lee, Vet Clin Small Anim, 43, 2013, 757-771; the disclosure of which is incorporated by reference.) Additionally, emesis induction is inconsistent in terms of gastric decontamination, as studies show that emesis attempts at home with hydrogen peroxide result in the expulsion of toxin 60% of the time; however when it does result in toxin expulsion it only results in expulsion of 45% of the toxin (See, e.g., Khan, S. A., et al., Effectiveness and adverse effects of the use of apomorphine and 3% hydrogen peroxide solution to induce emesis in dogs. Journal of the American veterinary medical association, 2012, 241(9), 1179-1184; the disclosure of which is incorporated by reference.) These findings are similar to other findings in human and veterinary medicine that call into question the effectiveness of emesis agents for decontamination (See, e.g., Tenenbein M, Cohen S, Sitar D S: Efficacy of ipecac-induced emesis, orogastric lavage, and activated charcoal for acute drug overdose. Ann Emerg Med August 1987; 16:838-841; Arnold Jr, F. J., Hodges Jr, J. B., Barta Jr, R. A., Spector, S., Sunshine, I., & Wedgwood, R. J. (1959); Evaluation Of The Efficacy Of Lavage and Induced Emesis In Treatment Of Salicylate Poisoning. Pediatrics, 23(2), 286-301; Buseman, M., A. E. Blong and R. A. L. Walton (2022); A. L., & Chin, L., Successful management of severe carprofen toxicity with manual therapeutic plasma exchange in a dog, J Vet Emerg Crit Care (San Antonio) 32(5): 675-679); Picchioni, (1972). Induced emesis—A questionable procedure for the treatment of acute poisoning. American Journal of Health-System Pharmacy, 29(10), 877-879); the disclosures of which are incorporated by reference.)

Activated carbon (or activated charcoal) can be used as a gastrointestinal adsorbent in animals. To be most effective, activated carbon (as well as other decontamination techniques) should ideally be given as soon as possible after toxic ingestion. In veterinary medicine, administration shortly after intoxication is almost impossible because of driving time (to the clinic), lapsed time since ingestion, time to triage, and the amount of time it takes to physically deliver activated charcoal (e.g., syringe feeding, orogastric tube). As a result, administration of activated charcoal is often delayed up to an hour or more. In addition, limitations of activated charcoal include its high volume needed to achieve affects, its unpalatable nature, its tendency to stain all that it touches, its potential to cause gastrointestinal impaction due to its fine physical nature, and its difficulty administering to animals in a veterinary and home environment. Furthermore, activated charcoal traditionally requires coadministration with liquids due to the unpalatable nature and physical characteristics of charcoal. These characteristics that make charcoal difficult to administer and potentially unsafe in the hands of pet owners, practically confine the use of activated charcoal to the hospital setting. Administration of activated charcoal products at home is not accepted for pet intoxication according to veterinary guidelines. Current veterinary guidelines do not recommend activated charcoal products for detoxification. (See, e.g., Avau B, et al., Cochrane Database of Systematic Reviews, 12, 2018, CD013230; G. K. Isbister, et al., Current Opinion in Critical Care, 17: 351-357, 2011; T. Zellner, et al., Dtsch Arztebl Int, 116: 311-317; 2019; the disclosures of which are incorporated by reference.) The inventors made the inventive realization that appropriate activated charcoal products can be beneficial for home use to detoxify intoxicated pets in a timely manner. However, existing activated charcoal products are not able to be used directly for detoxification purposes due to the safety and logistical concerns discussed. There is a significant need for detoxifying agents, including activated charcoal/carbon, in a consumer-friendly, household setting.

Many embodiments implement activated carbon for the detoxification of intoxications. Many embodiments provide methods to detoxify individuals, including mammals, humans, research animals, domesticated animals, livestock, and household pets (e.g., dogs, cats, birds, rodents, horses, cattle, and/or sheep) from toxic substances. Some embodiments use the activated carbon compounds for detoxifying dogs and cats. Some embodiments use the activated carbon compounds for detoxifying humans. Many embodiments are directed to compositions including activated carbon that can be used to detoxify toxins and other intoxications in an individual. Many embodiments may use activated carbon spheres for detoxification. In certain embodiments, the activated carbon compounds comprise activated carbon spheres created from a resin matrix. In some embodiments, the activated carbon compounds comprise the activated carbon spheres created from (but not limited to) coal, asphalt, polymers, lignin, carbohydrates, and other substances. In various embodiments, activated carbon is preferred to activated charcoal because activated carbon has higher purity, higher density, consistent physical characteristics, and higher quality. Many embodiments of compositions can be administered orally. In several embodiments, the activated carbon sphere compounds can be administered orally for detoxification of an intoxicated individual. In numerous embodiments, the individual is a human and/or a non-human animal, such as a household pet, including dogs and/or cats.

In many embodiments, activated carbon particles are created from a non-toxic matrix. In many embodiments, the final activated carbon spheres are clean, tasteless, easy to administer orally, and can be created from resin or any other acceptable compound. In this form, many embodiments resolve the issues with traditional activated charcoal, including (but not limited to) reducing volume needed, improving shelf-life, improving palatability, improving safety, and/or eliminating staining. In several embodiments, various flavors and/or food additives can be added to the matrix for easier administering. Additionally, such embodiments can be stored and administered in a home setting, thus minimizing the time between treatment and consumption of a toxin, that is inherently longer should the individual need to go to a hospital.

In many embodiments, the compounds can be formed as spheres, to optimize surface to volume ratio. Spherical particles have a high adsorption capacity, allowing them to effectively bind with toxins, carry them safely through the gastrointestinal (GI) tract and eliminate them from the individual's body in the feces without binding or damage to tissues. With one or more of the above characteristics, compounds containing activated carbon in accordance with many embodiments are at least 3 times; or at least 4 times; or at least 5 times; or at least 6 times, more effective by volume than traditional activated charcoals at removing toxins from humans and/or non-human animals.

### Activated Carbon Spheres

Activated carbon spheres (ACSs) are a type of activated carbon developed by Japan, the United States, Germany, and the Soviet Union in the late 1970s and gradually entered the industrialization stage in the 1980s. ACSs have advantages, including (but not limited to) porous structure, large specific surface area, high microporous volume, controlled pore size distribution, high mechanical strength, high purity, smooth surface, high wear resistance, excellent durability, good fluidity, low ash content, and low water content. ACSs have been used in dialysis and/or other, external to the body, purification systems.

Coal, asphalt, resins, polymers, lignin, carbohydrates, etc. can be utilized as precursors to prepare ACS using carbonization and activation processes. Various parameters, such as carbonization temperature, rising rate of temperature, activation time, activation temperature, activator, different activation methods, etc., can affect the performance and quality of the final product. At various sizes and shapes, ACSs have a wide range of roles for the removal of heavy metals, organic dyes, carbon dioxide, volatile organic chemicals, or as catalyst carriers, or for gas and energy storage, or as chemical protection materials, or for blood purification. ACSs can be used for hemodialysis to adsorb toxins in the blood. Conventionally, the ACSs can be used outside of the body in hemodialysis environments. ACSs and/or ACS compounds that can be administered orally are lacking for detoxification purposes.

Resin can be used as precursor materials for ACS. Resin has advantages such as high carbon content, no ash, and uniform molecular structure, which are not possessed by coal, asphalt, and cellulose. The resin-based ACS precursors that have been reported in the literature mainly include phenolic resins (PF), polyacrylonitrile (PAN), polyvinyl chloride (PVC), polyimide (PI), and poly(styrene-co-divinylbenzene) (PSDVB). Among them, polystyrene (PS) and PF have received great attention.

ACSs can be used for detoxifying similar functions as traditional activated charcoal, including to detoxify an individual's consumption (or suspected consumption) of an intoxicant. Additionally, ACSs, like traditional charcoals, may be able to disrupt enterohepatic circulation (or recirculation), where certain toxins (e.g., aspirin and certain other NSAIDs) are conjugated to glucuronic acid in the liver, excreted into bile, metabolized back into the free toxin by intestinal bacteria. The toxin is then reabsorbed into plasma. Via the recirculation into the intestine, ACSs have the potential to disrupt enterohepatic circulation. Furthermore, ACSs resolve, limit, or otherwise mitigate many of the limitations with traditional activated charcoal, such as bowel obstruction, staining, and difficulty in administering, among others.

### Activated Carbon Compounds

The activated carbons are processed (or activated) to have small, low-volume pores that can greatly increase the surface area available for adsorption or chemical reactions (depending on the adsorbate). Adsorption is a physical process where atoms or molecules adhere to surfaces of a material. Activation renders the activated carbons with a high surface-area-to-volume ratio. In several embodiments, activated carbon can be incorporated into compounds. The compounds in accordance with some embodiments can include activated carbon. Many embodiments implement activated carbon sphere (ACS) compounds for detoxification of humans and/or non-human animals. In this disclosure, activated carbon includes various types of activated carbon including (but not limited to) ACSs, unless specified otherwise. In this disclosure, compounds refer to activated carbon compounds and/or ACS compounds, unless specified otherwise. The active ingredients of the compounds refer to any type of activated carbon including (but not limited to) ACSs. In some embodiments, the compounds can include a variety of additives and/or impurities in addition to the active ingredients.

In many embodiments, the activated carbon is of high purity and quality, ensuring the safety and effectiveness of the compounds. Precursors such as (but not limited to) resin matrix can be used as the basis of the activated carbon. In several embodiments, the activated carbon can be used directly as the final compounds. In some embodiments, additives can be optionally added to the activated carbon to form the final compounds. The compounds can be molded into desired shapes and sizes. Such shapes and sizes can make consumption by an individual easier.

In several embodiments, activated carbons can have various shapes and sizes to achieve optimal detoxification effectiveness in humans and/or non-human animals. In some embodiments, the activated carbons can have a shape with one or more rounded corners. The rounded corners can increase the surface area to enhance adsorption. In some embodiments, the activated carbons can be in the form of powders, granules, particles, grains, beads, spheres, crystals, ovals, cylinders, and any combinations thereof. As can be readily appreciated, the activated carbons can have any shape that contains rounded corners as appropriate to the requirements of specific applications in accordance with various embodiments of the invention. Several embodiments implement ACSs as the final compounds. The spherical shapes of ACSs can have an advantageous adsorption profile and they may not damage tissues or cause other mechanical issues. In many embodiments, the activated carbons can have a porous structure with an optimal porosity to improve the surface area to volume ratio or target for specific adsorbates. In certain embodiments, the activated carbons such as (but not limited to) ACSs can have an average diameter from about 20 microns to about 100 microns; or from about 100 microns to about 500 microns; or from about 500 microns to about 600 microns; or from about 500 microns to about 700 microns; or from about 500 microns to about 800 microns; or from about 500 microns to about 900 microns; or from about 500 microns to about 1000 microns. In some embodiments, the activated carbons such as (but not limited to) ACSs can have an average diameter greater than or equal to about 1000 microns. In certain embodiments, the activated carbons can have an average diameter from about 1 millimeter to about 4 millimeters. In several embodiments, the sizes of the activated carbon can be selected for the optimal use in the compounds. In various embodiments, the activated carbons such as (but not limited to) ACSs can have high purity. In some embodiments, at least about 99 wt % of the activated carbons are carbon; or at least about 98 wt % of the activated carbons are carbon; or at least about 97 wt % of the activated carbons are carbon; or at least about 96 wt % of the activated carbons are carbon; or at least about 95 wt % of the activated carbons are carbon. The activated carbons can be activated via chemical processes or physical processes. In certain embodiments, physical processes such as (but not limited to) heating can be used to activate the activated carbon and remove any impurities during the manufacturing processes.

In several embodiments, polymers such as (but not limited to) resin matrix can be used as precursors to form the activated carbon. Examples of suitable materials for the resin matrix include (but are not limited to) polyurethane, epoxy, polyester resins, and any combinations thereof. During the activation step, the polymers can be removed to form pure activated carbon.

In many embodiments, the activated carbon can be used directly as the activated carbon compounds for detoxification in humans and/or non-human animals. Some embodiments use the ACSs as the activated carbon compounds for detoxification in humans and/or non-human animals. The dosage of the activated carbon in the compounds can be optimized as appropriate to the requirements of specific applications in accordance with various embodiments of the invention.

Several embodiments of a compound further utilize other components and excipients. For example, flavors, flavor enhancers, buffering agents, thickeners, stabilizers, and/or plasticizers to make the dosage form more palatable. In many embodiments, the components are non-reactive with the compounds to maintain efficacy of the compounds during storage. Common flavoring agents and flavor enhancers that may be included in the formulation of the present invention include, but are not limited to, fruit (apple, orange, berry), mint, chocolate, beef, chicken, bacon, tuna, peanut butter, cheese, and any other flavors.

In many embodiments, the compounds can be molded into various shapes and sizes for easy intakes of non-human animals. In some embodiments, the compounds can be molded in the form of granules, particles, grains, beads, spheres, ovals, cylinders, and any combinations thereof. In several embodiments, the compounds can be molded in the form of polyhedrons, cubes, cuboids, triangles, tetrahedrons, bone shapes, star shapes, fish shapes, bow-tie shapes, and any combinations thereof. As can be readily appreciated, the compounds can have any form as appropriate to the requirements of specific applications in accordance with various embodiments of the invention. In several embodiments, the compounds can have at least one dimension from about 20 microns to about 100 microns; or from about 100 microns to about 500 microns; or from about 500 microns to about 600 microns; or from about 500 microns to about 700 microns; or from about 500 microns to about 800 microns; or from about 500 microns to about 900 microns; or from about 500 microns to about 1 millimeter; or greater than or equal to about 1 millimeter; or greater than or equal to about 2 millimeters; or greater than or equal to about 3 millimeters; or greater than or equal to about 4 millimeters; or greater than or equal to about 5 millimeters. In many embodiments, the compounds can have an average diameter from about 20 microns to about 100 microns; or from about 100 microns to about 500 microns; or from about 500 microns to about 600 microns; or from about 500 microns to about 700 microns; or from about 500 microns to about 800 microns; or from about 500 microns to about 900 microns; or from about 500 microns to about 1 millimeter; or from about 1 millimeter to about 5 millimeters; or from about 5 millimeters to about 10 millimeters. In various embodiments, the compounds can be a mix of a variety of physical characteristics. In various embodiments, the compounds can include at least about 50% of activated carbon; or from about 50% to about 100% activated carbon. In some embodiments, the compounds can include at least about 60% activated carbon. In some embodiments, the compounds can include at least about 70% activated carbon. In some embodiments, the compounds can include at least about 80% activated carbon. In some embodiments, the compounds can include at least about 90% activated carbon. In some embodiments, the compounds can include 100% activated carbon. In some embodiments, the compounds can include 100% ACSs.

### Products Containing Activated Carbon

Certain embodiments provide activated carbon compounds as kits, products, another item of manufacture and combinations thereof. Such embodiments can include activated carbon compounds provided as aliquots measured for a specific dose or use.

Certain embodiments provide a vessel (e.g., container) containing activated carbon compounds. Some embodiments may include ampules, vials, jars, bottles, envelopes, sachets, and/or other storage vessels containing activated carbon compounds. In certain embodiments, a vessel includes a specific number of doses that can be used (e.g., 1 dose, 2 doses, 3 doses, 4 doses, 5 doses, 10 doses, etc.). In some embodiments, the doses may be based on mass of activated carbon compounds (e.g., 50 g, 100 g, 150 g, 200 g, 250 g, etc.). In some embodiments, the doses may be for a particular size of the individual to receive the dose (e.g., 515 lbs., 16-30 lbs., 31-45 lbs., 46-100 lbs., 100-150 lbs., 150-200 lbs., 200 lbs., etc.). Such embodiments may include lines, graduations, demarcations, or other marking to assist in dosing or portioning activated carbon compounds. In certain embodiments, a single vial can include markings and/or instructions for a particular dose (e.g., 1 dose, 2 doses, 15 grams, 30 grams, pets ≤15 lbs., pets 16-30 lbs., etc.). Table 2 provides another non-limiting example of dosing instructions based on weight of a pet (e.g., dog or cat) as measured by number of vials and mass of activated carbon compounds. In certain embodiments, a single vial can include markings and/or instructions for a particular dose for a human subject (e.g., 1 dose, 2 doses, 100 grams, 200 grams, subjects 100 lbs., subjects 100-150 lbs., etc.).

In some embodiments, the products containing activated carbon compounds can be stored dry or desiccated. In some embodiments, the products containing activated carbon compounds can be stored in a medium such as (but not limited to) water, aqueous solution, ionic solution, gel. In embodiments to be consumed, the media is preferably non-toxic. Such media may include buffers, flavors, thickeners, stabilizers, plasticizers, etc. In many embodiments, the media is non-reactive with activated carbon compounds to maintain efficacy of the activated carbon compounds during storage.

Certain embodiments are packaged as treats, snacks, or other ingestible forms. Such embodiments may be easier to provide to an individual due to an increased pleasure of consuming. Some embodiments are dog treats or snacks, such as beef, bacon, peanut butter, cheese, chicken, and/or any other flavor treat or snack that includes activated carbon compounds within the snack. Exemplary snacks or treats include dry treats (e.g., dog biscuits). Further embodiments are wet or gelatinous treats or snacks, for example, a squeezable gel treat. Squeezable treats or snacks can be provided directly from a container, provided on a dish (e.g., bowl, plate, etc.), and/or mixed with food.

Certain embodiments are packaged as foods, treats, snacks, condiments, additives, beverages, or other ingestible forms for humans. Some embodiments are powders, bars, syrups, gels, sauces, beverages, and/or any other forms of food that includes activated carbon compounds.

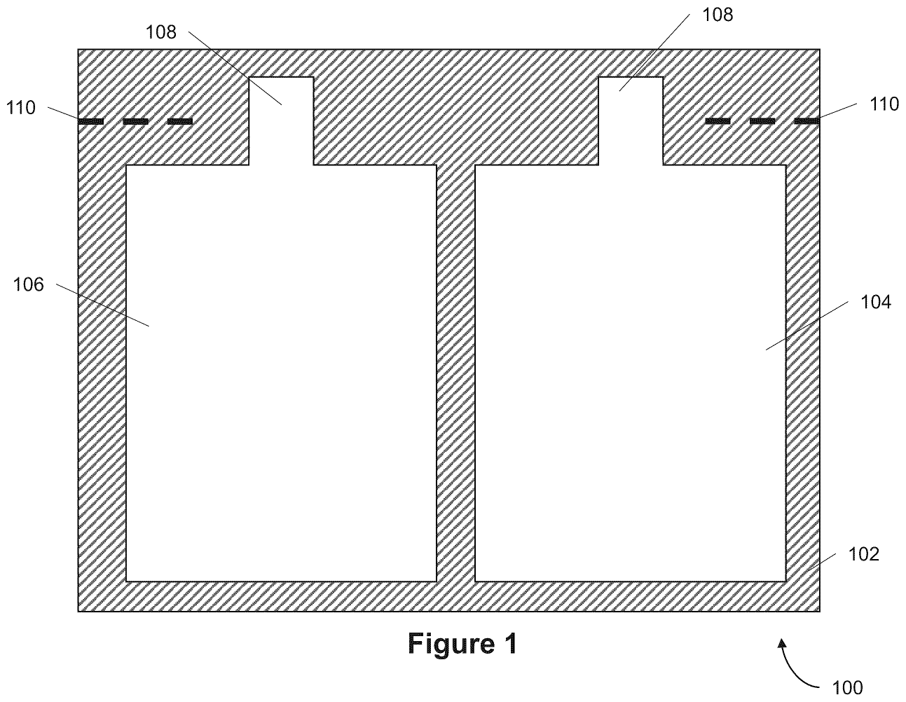

Certain embodiments are packaged as a post-mix product, such that one container contains activated carbon compounds (dry or in media), while a second container includes a mixing agent to assist with providing or administering the activated carbon compounds to an individual. The second container may include a solid, liquid, gel, or other agent to assist. Some mixing agents can be flavored, such as described above. Various post-mix embodiments may be packaged as a multi-part envelope, similar to candies such as Fun Dip and/or Lik-M-Aid. FIG. 1 provides a non-limiting example of a multi-part envelope or packaging 100. In the illustrated example, packaging 100 can be formed of a material 102 that is sealed around its perimeter to form pouches 104, 106. Given that some embodiments may include a liquid or gel, the pouches can be made from coated paper, plastic, foil, mylar, and/or any other material suitable for maintaining efficacy of such products while preventing accidental breach, tear, or puncture of the packaging 100. In certain embodiments, packaging material can be biodegradable. Biodegradable packaging may be beneficial or preferable during camping, hiking, or other outdoor activities, where convenience and environmental stewardship are desirable.

A first pouch 104 may contain the activated carbon compounds, such as described herein, while a second pouch 106 may contain a medium to assist in consumption. Pouches 104, 106 may be of any size to hold a desired volume of the activated carbon compounds and/or medium. In various embodiments, material 102 is sealed to form a narrowing 108 of pouches 104, 106. Such a narrowing may assist with dispensing the contents of pouches 104, 106. Additional embodiments include a perforation 110 or other feature to assist with opening packaging 100.

Various embodiments are directed to kits that include the activated carbon compounds. Such kits can include one or more of the foregoing products. Certain embodiments of kits further include a container including a substance that may ease administration, such as an edible ingredient (e.g., peanut butter, water, broth, food, etc.). Some embodiments may include a measuring device (e.g., scoop, spoon, etc.), a mixing device (e.g., stick, spoon, etc.), a dish for mixing and/or serving (e.g., bowl, plate, etc.). Certain embodiments of kits include a case, box, or bag, in which components are packaged.

### Methods of Use

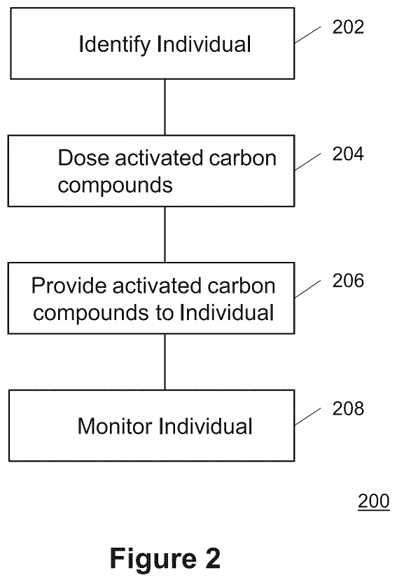

In a number of embodiments, activated carbon compounds are administered to an individual (including a household pet such as dogs, cats, or other small mammals) that have ingested toxic substances. The activated carbon binds with the toxins in the digestive tract to prevent the toxins from being absorbed into the bloodstream. The bound toxins can then be eliminated from the body through excrement (e.g., feces). Adsorption of the adsorbate (toxin) to the activated carbon occurs through van der Waals forces rather than chemical binding. FIG. 2 provides an example of a method for providing the activated carbon compounds to an individual. In many embodiments, the individual is a mammal. In some embodiments, the individual is a human. In several embodiments, the individual is a domesticated animal. In various embodiments, the individual is a non-human animal. In certain embodiments, the individual is a household pet (e.g., dog, cat, etc.).

At 202, many embodiments identify or have identified an individual that has consumed or is suspected of consuming a toxic or potentially toxic substance. In certain embodiments, the toxic substance is a common, medical, household, natural, and/or any other product. As noted previously, an exemplary and non-limiting list of such substances is provided in Table 1. Identification can be based on behavioral, symptomatic, or evidentiary information. For example, behavior information can include whining, pacing, panting, crying, and/or any other abnormal behavior. Symptomatic information can include pain, vomiting, diarrhea, constipation and/or other abnormal health conditions. Evidentiary information can include other information indicating possible consumption of an intoxicant, such as spilled substances, partially eaten items, empty wrappers, and/or any other evidence that a toxic or potentially toxic substance has been consumed. It should be noted that the foregoing lists of information types are only exemplary to demonstrate possible things that can be used to identify an individual. These examples are not meant to be limiting.

In many embodiments, the activated carbon compounds are dosed at 204. In various embodiments, dosing 204 includes measuring out a sufficient amount of activated carbon compounds to administer. This amount can be based on the amount of toxic compounds and/or based on a volume of activated carbon compounds/mass of the individual (e.g., the individual identified at 202). In embodiments, where activated carbon compounds are pre-mixed (e.g., made into a treat or snack), dosing can include counting the amount of snacks or treats containing enough activated carbon compounds for a particular size of individual (e.g., each treat is sufficient for up to 15 pounds of mass).

In post-mix embodiments, dosing 204 includes mixing the activated carbon compounds with a substance to ease consumption of the activated carbon compounds. Such mixing can include mixing ACSs with substances to ease consumption, such as an edible substance. Such substances can include certain nut butters (e.g., peanut butter, almond butter), a liquid (e.g., water, broth, beverage, etc.), wet food, softened food (e.g., dry kibble mixed with water or another liquid), canned tuna, potted meat, and/or any other edible substance. Additional embodiments include an edible and/or potable thickener to assist in consumption, such as to prevent activated carbon compounds from sinking and/or to assist in swallowing, if such reflexes are weakened or dampened due to intoxication. A non-limiting example of an edible thickener is Thick-It.

A dose of activated carbon compounds is provided to an individual at 206, in accordance with many embodiments. In many such embodiments, the dose was prepared (e.g., measuring and/or mixing an amount of activated carbon compounds) at 204. Additionally, the individual can be the individual identified at 202.

Depending on the dose preparation, the dose activated carbon compounds can be provided to the individual for voluntary consumption, such as when the individual takes the dose of activated carbon compounds without assistance. In certain embodiments, the dose of activated carbon compounds is forcibly provided via forced consumption, such as by gastric intubation, gavage, and/or any other relevant process to get the dose of activated carbon compounds into the stomach of the individual. Forced consumption may be necessary when the individual is unable or unwilling to consume the dose of activated carbon compounds themselves and would be performed in the veterinary setting.

The individual is monitored at 208 of many embodiments. Such monitoring is to determine efficacy of detoxification and can include identifying any changes in symptoms and/or behavior, such as the non-limiting examples listed in regard to 202. Ideally, changes in the symptoms and/or behavior are to improve and/or return to normal. Monitoring 208 can extend until such time that symptoms and/or behavior return to normal (e.g., non-intoxicated behavior).

However, if intoxication is too great, symptoms and/or behavior may not change, may worsen, or only improve slightly. In such circumstances, various embodiments may repeat dosing activated carbon compounds 204, providing a dose of activated carbon compounds 206, and monitoring the individual 208 until symptoms and/or behavior improve and/or return to normal. Repeat dosing can occur at various intervals as appropriate for size of the individual and/or severity of intoxication. Generally, each dose can occur approximately (e.g., ±15 minutes) 2-4 hours apart (e.g., ˜2 hours, ˜2.25 hours, ˜2.5 hours, ˜2.75 hours, ˜3 hours, ˜3.25 hours, ˜3.5 hours, ˜3.75 hours, or ˜4 hours). As noted, depending on size or severity, dosing can occur more frequently (e.g., <2 hours) or at greater intervals (e.g., >4 hours).

It should be noted that the method 200, described and illustrated herein, is meant as a non-limiting example of methods to provide and/or administer activated carbon compounds to an individual. Certain embodiments may include additional features, omit certain features, repeat certain features, and/or perform features in a different order (including simultaneously).

### EXEMPLARY EMBODIMENTS

Although specific embodiments of systems and apparatuses are discussed in the following sections, it will be understood that these embodiments are provided as exemplary and are not intended to be limiting.

### Example 1: Efficacy of the Activated Carbon Compounds

Several embodiments evaluate the adsorptive capacity of the activated carbon compounds and compare the compounds with three different resin based activated carbon, a laboratory grade activated charcoal powder, and the veterinary standard charcoal Toxiban (containing Med Char).

A variety of toxins such as acetaminophen, methylxanthine, THC, xylitol, carprofen, ibuprofen, amphetamines, naproxen, caffeine, antifreeze, ethylene glycol, can be tested. In some embodiments, about 200 mg acetaminophen can be added to a fixed quantity of 4 different activated carbon resins (1 g), 1 type of activated charcoal commonly used in clinical settings (MedChar) and 1 type of pure activated charcoal. Each admixture was agitated for 5 minutes, incubated at body temperature (e.g., 38.5° C.) for about 1 hour and then centrifuged for about 30 minutes. The concentration of residual, non-adsorbed acetaminophen in the supernatant was quantitatively assayed by reverse phase high-pressure liquid chromatography with ultraviolet detection. 2 sample T-test can be used to determine statistical significance with statistical significance set at P<0.05. Additional impurities in the three different forms of carbon were tested.

The study was conducted using commercially available Pure Activated Charcoal, Toxiban Granules (50% MedChar by weight), four different activated carbon resins, as well as commercially available acetaminophen (Calpol Infant Suspension (120 mg/5 mL/L). Tested samples include commercially available activated charcoal that can be used as positive control, Toxiban granules, the exemplary compound in accordance with embodiments, Resin 2—Other 1, Resin 3—Other 2, Resin 4—Other 3. The Resin 2, Resin 3, and Resin 4 test samples result in ACSs that have different pore size, surface areas, densities, and gross physical size in comparison with the exemplary compound. Max toxin adsorption between Toxiban, the exemplary compound and test samples at the acetaminophen dose is tested, using g/g basis. Toxiban granules (as this is a 500 mg/g of charcoal) can be used as positive control. About 2 g Toxiban granules were used.

About 200 mg of acetaminophen (8.4 mL) was placed into a sterile centrifuge tube and diluted with 10 mL distilled water. About 1 g of activated charcoal equivalent (2 g of granules and 1 g of test carbon resin) was then introduced to the centrifuge tube containing the diluted acetaminophen solution. The samples were manually agitated for 5 minutes to ensure thorough mixing and then incubated at 38.5° C. for 1 hour, while being gently agitated at 1.7 Hz in a shaker bath. The samples were then immediately centrifuged for 30 minutes at 2400×g and 38° C. The resultant supernatant was siphoned off and evaluated for quantitative acetaminophen analysis by reverse phase high-pressure liquid chromatography (H PLC) with ultraviolet detection. No special transport media is required because acetaminophen is stable in suspension. Two separate samples were analyzed from each admixture.

| | Negative | Activated | Toxiban | Exemplary | Resin 2 | Resin 3 | Resin 4 |
|---|---|---|---|---|---|---|---|
| Sample | Control | Charcoal | Granules | compound | (R2) | (R3) | (R4) |
| 200 mg | NC.200.1 | AC.200.1 | T.200.1 | R1.200.1 | R2.200.1 | R3.200.1 | R4.200.1 |
| sample 1 | | | | | | | |
| 200 mg | NC.200.2 | AC.200.2 | T.200.2 | R1.200.2 | R2.200.2 | R3.200.2 | R4.200.2 |
| sample 2 | | | | | | | |

Tests for the purity of the samples can be conducted via Residue on Ignition Testing. This procedure is used for the determination of the residual substance not volatilized from sample. Following USP<281> (US Pharmacopeia): The Residue on Ignition/Sulfated Ash test utilizes a procedure to measure the amount of residual substance not volatilized from a sample when the sample is ignited in the presence of sulfuric acid according to the procedure described below. The test is used for determining the content of inorganic impurities in an organic substance. Test results show that the exemplary compound is the purest in terms of inorganic substances by 2-4 times when compared to other carbon resins.

Thermogravimetric analysis. The weighted sample was put in the oven. The heating temperature was 25° C. under simulated ambient conditions. The starting temperature was 25° C. and heated to 600° C. (30 mins). Then, the temperature was held at 600° C. for 1 hr. After 1 hr, the residue was then weighed and calculated.

| Sample | % Residue (inorganic compounds) |
|---|---|
| Exemplary compound | 0.13% |
| Resin 2 | 0.23% |
| Resin 3 | 0.40% |
| Resin 4 | 0.48% |

Test results show that on a per weight basis, the 4 activated carbon spheres that were evaluated were equally proficient in adsorbing acetaminophen. When efficacy was converted to a per volume basis, efficacy of acetaminophen binding per ml of the exemplary compound was at least 3.4 times of the binding capacity of the Toxiban granules.

| | Results by Weight | | | | |
|---|---|---|---|---|---|
| | | Absorption of | | | |
| | | Acetaminophen | | | Relative |
| | % Absorption | per Gram of | Results by Volume | | Effectiveness |
| | of 200 mg | Charcoal | Density | Absorption | compared to |
| Sample | Acetaminophen | Equivalent | (g/ml) | (mg/ml) | Toxiban |
| Activated | 92% | 185.4 mg | 0.32 | 59 | 2.0 |
| Charcoal | | | | | |
| Toxiban | 93% | 183.5 mg | 0.16 | 29.2 | 1 |
| (granules) | | | | | |
| Exemplary | 91% | 181.5 mg | 0.55 | 99.0 | 3.4 |
| compound | | | | | |
| Resin 2 | 94% | 187.4 mg | 0.33 | 62.3 | 2.1 |
| Resin 3 | 89% | 177.2 mg | 0.29 | 52.2 | 1.8 |
| Resin 4 | 94% | 187.6 mg | 0.29 | 55.3 | 1.9 |

### Doctrine of Equivalents

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

As used herein, the singular terms “a,” “an,” and “the,” may include plural referents unless the context clearly dictates otherwise. Reference to an object in the singular is not intended to mean “one and only one” unless explicitly so stated, but rather “one or more.”

As used herein, the terms “approximately” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to +0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. Where ranges are described, the range should be understood to include the endpoints of the ranges, and the endpoints of such ranges are also contemplated to stand on their own as inventive, individual data points and to form the endpoints of other ranges. For example, a ratio in the range of about 1 to about 200 should be understood to include the explicitly recited limits of about 1 and about 200, but also to include individual ratios such as about 2, about 3, and about 4, sub-ranges such as about 1 to about 10, about 10 to about 50, about 20 to about 100, about 100 to about 200, and so forth, and related ranges such as greater than about 1 or less than about 200.

TABLE 1
Non-Limiting List Common Intoxicants

Human Medications
Pain medications
Antidepressants
Heart medications
ADHD Medications (amphetamines)
Cannabis products (THC, CBD, etc)
Over-the-counter products

| | |
|---|---|
| NSAIDS | |
| | Ibuprofen |
| | Naproxen |
| | Acetaminophen |
| | Aspirin |
| | Diclofenac |
| Human food | |

garlic and onions

n-propyl disulfide and 3 sodium alk(en)yl thiosulfates
sodium n-propylthio-sulfate

grapes and raisins

Mechanism of action are unknown (thought to be ochratoxins (mycotoxins),
pesticides, tannins, or monosaccharides)

Moldy foods (Mycotoxins)

Aflatoxins
Tremorogenic Mycotoxins

Veterinary medications
Chewable medications (Carprofen)

Chocolate

Methylxanthine

Theobromine
Caffeine

Household toxicants

Bleach

Cleaning supplies
Insecticides (flea control spot-ons)

Amitraz

Metaldehyde
Imidacloprid
Organophosphates and Carbamates
Pyrethrins and Pyrethroids

Rodenticides

Bromethalin
Cholecalciferol
Anticoagulants
Difethialone
Brodifacoum
Bromadiolone

Warfarin

Chlorophacinone
Diphacacinone

Plants

Lilies (Lilium and Hemerocallis sp)

Specific toxin has not been identified but is water soluble

Sago palms (Cycas revoluta)

Azoxyglycosides - Cycasin, macrozamin, neocycasin
Beta-Methylamino-1-alanine

Oleander (Nerium oleander)
Mushrooms and mycotoxins

Herbicides

Certain outdoor products

TABLE 2
Example Dosing Instructions

| | | | activated carbon |
|---|---|---|---|
| Weight Range (lbs.) | # of Vials | | compounds (g) |
| Dogs/Cats ≤15 lbs. | ½ | vial | about 15 |
| Dogs 16-30 lbs. | 1 | vial | about 30 |
| Dogs 31-60 lbs. | 2 | vials | about 60 |
| Dogs 61-90 lbs. | 3 | vials | about 90 |
| Dogs 91-150 lbs. | 5 | vials | about 150 |

What is claimed is:

1. A method to detoxification and gastrointestinal decontamination, comprising:
dosing an amount of activated carbon compounds; and
providing the dosed amount of activated carbon compounds to an individual, wherein the individual is a mammal.

2. The method of claim 1, wherein the activated carbon compounds comprise activated carbon spheres.

3. The method of claim 1, wherein the activated carbon compounds consist of 100% activated carbon spheres.

4. The method of claim 1, wherein the individual is a human or a household pet.

5. The method of claim 1, wherein the individual is a dog or cat.

6. The method of claim 1, wherein the individual has consumed or is suspected of consuming or having consumed a toxic substance.

7. The method of claim 6, further comprising identifying the individual based on one or more of behavioral, symptomatic, and evidentiary information.

8. The method of claim 7, wherein:
the behavioral information includes one or more of: whining, pacing, panting, and crying;
the symptomatic information includes one or more of: pain, vomiting, diarrhea, and constipation; and
the evidentiary information includes one or more of: spilled substances, partially eaten items, and empty wrappers.

9. The method of claim 6, wherein the toxic substance is selected from one or more intoxicants in Table 1.

10. The method of claim 1, wherein dosing an amount of activated carbon compounds comprises measuring an amount of activated carbon compounds to provide to the individual.

11. The method of claim 10, wherein dosing an amount of activated carbon compounds further comprises mixing the measured amount of activated carbon compounds with a substance to ease consumption.

12. The method of claim 11, wherein the substance is selected from: food, drink, peanut butter, canned food, water, broth, and softened food.

13. The method of claim 1, wherein providing the dosed amount of activated carbon compounds comprises forcibly providing the dosed amount to the individual by gastric intubation or gavage.

14. The method of claim 1, further comprising monitoring the individual for changes in behavior or symptoms.

15. The method of claim 14, further comprising:
if symptoms or behavior do not improve or if symptoms or behavior worsen, dosing a second amount of activated carbon compounds; and
providing the second dosed amount of activated carbon compounds to the individual.

16. A composition for detoxification, comprising a container including an amount of activated carbon compounds.

17. The composition of claim 16, wherein the activated carbon compounds comprise activated carbon spheres.

18. The composition of claim 16, further comprising an edible ingredient, in which the activated carbon compounds are mixed.

19. The composition of claim 18, wherein the edible ingredient is selected from the group consisting of: a dog biscuit, a wet treat, a gelatinous treat, a cat treat, a human food, a snack, and a beverage.

20. The composition of claim 16, wherein the container is a multi-part container forming a first pouch and a second pouch, wherein the first pouch includes the amount of activated carbon compounds, while the second pouch includes an edible ingredient.

21. The composition of claim 16, wherein the container includes a plurality of doses of activated carbon compounds.

22. The composition of claim 16, wherein the container comprises markings denoting individual doses of activated carbon compounds.

23. A detoxification kit, comprising:
a box packaging a container including an amount of activated carbon compounds.

24. The detoxification kit of claim 23, further comprising at least one of: a measuring device, a mixing device, and a dish.

25. The detoxification kit of claim 23, wherein the activated carbon compounds comprise activated carbon in a resin matrix.
